(19) United States
(12) Patent Application Publication
(10) Pub. No.: US 2026/0229345 A1
(43) Pub. Date: Aug. 6, 2026

(54) MACHINE VISION SYSTEMS AND METHODS FOR ROOM STATE DETERMINATION

(71) Applicant: SURGICAL SAFETY TECHNOLOGIES INC., New York, NY (US)

(72) Inventors: Filip GRANTCHAROV, New York, NY (US); Peter Dupont GRANTCHAROV, New York, NY (US); Po-Chieh LIU, New York, NY (US); Kee TANG, Toronto (CA); Amar CHAUDHRY, Toronto (CA)

(21) Appl. No.: 19/042,253

(22) Filed: Jan. 31, 2025

Related U.S. Application Data
(60) Provisional application No. 63/626,730, filed on Jan. 30, 2024.

(51) Int. Cl. G16H 40/20; G06V 10/30; G06V 10/771; G06V 10/774; G06V 10/82; G06V 10/94; G06V 20/40

(52) U.S. Cl. CPC G16H 40/20; G06V 10/30; G06V 10/771; G06V 10/7747; G06V 10/82; G06V 10/95; G06V 20/41

(57) ABSTRACT

A computer implemented approach using machine vision machine learning model architectures processing a stream of digital images obtained by physical sensors, such as cameras or recording equipment, for determining room states is proposed. The cameras or recording equipment operate in concert with a controller backend computing system that processes recordings made in one or a plurality of physical facilities, such as operating theatres, providing a healthcare “black box” recorder. A challenge with working with these recordings is that they are captured in a raw format and are both bandwidth and storage intensive. Predicted and estimated room states are then utilized for generating predictive outputs or improved scheduling.

### CROSS-REFERENCE

This application is a non-provisional of, and claims all benefit, including priority to U.S. Application No. 63/626,730, filed 30 Jan. 2024, entitled “MACHINE VISION SYSTEMS AND METHODS FOR ROOM STATE DETERMINATION”, incorporated herein by reference in its entirety.

### FIELD

Embodiments of the present disclosure relate to the field of machine vision and machine learning, and more specifically, embodiments relate to devices, systems and methods for room state determination using machine vision.

### INTRODUCTION

There is a desire to improve the efficiency and resource allocation related to healthcare service delivery. Healthcare facilities are equipped with sophisticated medical equipment, and are valuable, limited resources. Accordingly, improving the overall utilization rate and/or providing improved decision making related to physical healthcare facilities is important.

It was observed that the current scheduling of hospital operating rooms (OR) exhibits areas that could be improved, particularly regarding idle time and room utilization. To address these inefficiencies, the machine learning approach proposed herein leverages advanced optimization techniques, and the goal is to refine the existing daily hospital schedule using computational tools and machine vision, maximizing the productive use of operating rooms and minimizing periods of inactivity. By optimizing the scheduling process, the goal is to enhance overall operational efficiency, ultimately improving patient care and resource management within the hospital.

A challenge with room resources is that healthcare facilities require high availability and demand can be unpredictable, as different complications can arise, and new urgent cases may arise. Healthcare organizations are thus required to maintain a level of excess room inventory to handle unexpected situations, but the excess room inventory is costly as the rooms are equipped with expensive machinery and require effort to maintain and sanitize between uses. Delays have cascading effects, impacting availability and potentially requiring costly overtime. On the other hand, excessive idle time and uncoordinated idle time can lead to inefficiencies.

While some major hospital networks can have individuals operating as “traffic controllers” that manually assign and reorganize schedules, this is an expensive undertaking, and the proposed system described herein can be used as a special purpose computer device that controls logistical room scheduling automatically using processed machine vision inputs received from one or more cameras.

### SUMMARY

A machine vision system and corresponding computer processing equipment is set up to record room state video, and then process the room state video in real or near-real time to continually generate metadata in the form of timestamped predictions of whether the room is idle, in use, or in a turnover condition, among others.

The machine vision system and corresponding computer processing equipment can, in some embodiments, be part of, or operate in parallel with a “surgical black box” machine vision platform that couples different recording devices that are used to generate recording streams that are then time synchronized and used as an input for generating machine learning based insights automatically from recorded video.

By being able to estimate the room state based on the machine vision, a coupled machine learning engine and room schedule logistics controller can receive the room state as an input data set, and automatically update a data structure representing room allocations and scheduled events that require different rooms.

This data input allows for prospective scheduling that is optimized to accommodate one or more programmed outcomes, such as attempting to finish all procedures by a particular time, clearing a particular room for a long duration of time so that it can be opportunistically maintained or simply taken out of rotation to reduce costs, or to attempt to ensure that there is a sufficient level of spare capacity to handle unexpected events, such as a mass casualty event or significant levels of unexpected illness (e.g., mass food poisoning, unexpectedly heavy influenza spread).

A computer implemented approach using machine vision machine learning model architectures processing a stream of digital images obtained by physical sensors, such as cameras or recording equipment is proposed. In particular, machine learning approaches are proposed that are adapted to operate in a high-volume, resource (processing resources and processing time) constrained environment.

The approach can be scalable to handle multiple rooms at once, which allows a scheduler engine to opportunistically modify schedules as between different rooms by conducting different test insertions and deletions throughout a day in an attempt to dynamically modify schedules based on actual procedure timing as opposed to the original scheduled procedure timing. For example, a central scheduling system can be coupled to fifty different operating rooms, each with a local instance of the machine vision and room state estimation prediction.

The synchronized metadata can be provided in real or near-real time to the central scheduling system, which then can monitor real-world estimated room states, compare against a schedule, and re-arrange and reschedule bookings automatically. As described herein, matching room states and scheduled procedures is non-trivial as there can be technical issues that arise in separating out different cases, especially as scheduled procedures and their actual operating time can vary significantly as complications arise, etc., or new items are found during exploratory surgery. Conversely, some procedures may be straightforward and additional precautions taken during scheduling may not be necessary, and these procedures may end early. Accordingly, improved approaches are also proposed to provide an improved real-time or near-real time matching engine so that the room state predictive outputs can be practically used in dynamic calendaring/schedule modifications, which is a useful tool for continually optimizing towards minimizing of a specific loss function or objective function.

The room state outputs can thus be integrated into a practical tool that acts as an automatic traffic controller, continuously optimizing schedules and controlling room, device, and human resource allocations through controlling calendar invite and reservation data messages that can be “locked in”, for example, a few hours before the procedure, allowing the system to continuously and dynamically modify and test different simulated schedules as procedures complete ahead or behind schedule.

An example optimization may include configuring the system to, where possible, avoid using an entire specific operating room for a day, reserving it as excess capacity to handle a major unexpected event or to simply allow cost reduction by allowing it to be removed from usage for an entire day (significantly reducing overhead as it would not need to be set up at all for the day).

In some embodiments, the system includes a trained machine learning model that is configured to generate predictions of future states using inputs including at least historical surgical times for a particular surgeon, a type of procedure, the patient's EMR, and the generated predictions can then be used for generating test scheduling or schedule insertions to optimize a target outcome.

Accordingly, the system can be configured to periodically re-generate a new schedule or portions thereof based on deviations between scheduled, estimated actual room states, and estimated future room states. In some embodiments, the machine vision inputs are also used to estimate a type of procedure, as it may not always be clear in the EMR or the schedule what type or what variation of a particular procedure is being undertaken (e.g., open heart vs. laparoscopic valve replacement surgery), or there may be mistaken entries in the EMR or the schedule. The re-generated schedule can include time allocated for turnover and sanitization.

In some embodiments, the schedule across multiple days can be re-generated periodically to optimize a feature that can span multiple days. For example, the scheduling can be optimized such that a room can be designated as unused for an entire day, which can significantly save on operating costs and overhead costs through dynamic and opportunistic rescheduling of different elective surgeries and re-arrangement thereof of other surgeries.

As a further variation, different procedures may have different classifications (e.g., urgent surgery, elective surgery), and depending on load and availability, certain procedures may be automatically scheduled in (e.g., if there is unexpected availability) or moved earlier into a day, while conversely, the system may automatically delay certain procedures if there is no availability or not enough excess availability (e.g., an elective or non-urgent surgery may be automatically rescheduled if there has been a major complication on an existing surgical procedure and the room is suddenly no longer available, impacting downstream schedules). The machine vision approach proposed herein provides much more granularity to access an “on the ground” machine understanding of availability, and this is used to automatically conduct schedule modifications by the backend system. The schedule modifications can be conducted by an optimizer engine subunit of the system, which can be a central server coupled to a plurality of different rooms.

The cameras or recording equipment operate in concert with a controller backend computing system that processes recordings made in one or a plurality of physical facilities, such as operating theatres, providing a healthcare “black box” recorder. A challenge with working with these recordings is that they are captured in a raw format, and are both bandwidth and storage intensive.

It is advantageous to be able to utilize processed outputs from the recordings to be able to assist with logistical decision making. Accordingly, a computer implemented, machine learning approach is proposed that processes the recordings in real or near-real time such that logistical decision making can be supported through providing informational outputs on a user interface, or in some embodiments, logistical decision making is automated through invocation of secondary workflow processes that operate to re-arrange scheduling and bookings, such as room reservations and corresponding equipment setup in an attempt to optimize a particular outcome, such as an overall reduction in delayed cases, an overall reduction in when a last case is scheduled, etc.

The approach uses a multi-layered machine learning approach. The layers can include a first layer providing a deep learning model for processing an input image (e.g., a decrypted image), a second layer that conducts smoothing (e.g., using gradient boosting trees), and a third layer that is adapted to process the outputs of the first and second layer to conduct anti-flickering mechanisms to reduce the propensity of flicker states.

In some embodiments, the machine room vision can be obtained from a same camera and recording system as a surgical black box system, but to maintain a speed of determination, the room state determination can be conducted using a local computing system residing in or proximate to the room and dedicated for room state analysis for the particular room, while the recordings are also being made available to a slower centralized machine estimation backend that is configured for conducting black box type determinations and estimations, such as estimating surgical errors or other events that require a deeper level of machine learning analysis and more computing resources.

A room state determination is ultimately conducted by the machine learning model, and this can be captured in the form of a specific machine learning output, and the room state for a particular room can thus be determined. Example room states can include unoccupied, room setup, occupied-scheduled procedure, occupied-procedure requiring more time than allotted, room de-provisioning, etc.

The room states are based on machine learning outputs based on processing of raw surgical recordings. Because there can be a large number of recordings being processed at any given time, bandwidth and processing constraint considerations are important. Specific approaches are also proposed in various embodiments to assist in reducing computing burden in an attempt to provide outputs within a desired timeframe, even at a cost of accuracy.

The room state output can be provided in the form of a decision support interface, such as a portal for an administrator who is able to view existing room states and downstream estimated room states or expected delay metrics based on the room states. The administrator, in some embodiments, may also provide corrections to the room states for future re-training of the model.

In some embodiments, the system is also configured to automatically determine one or more future room state configurations to be proposed as candidate options for selection by the administrator, who can implement the room state configurations for future scheduling through selecting from a series of options being presented. In another embodiment, the system can automatically operate and re-arrange and dynamically schedule operations for the future based on the existing room state and/or determinations of future room state.

In some embodiments, room state modifications for future estimations are conducted using a machine learning model that takes into account previous room state determinations associated with a particular room or surgical team, adding time modifications (e.g., expected delays). For example, a particular surgeon may be very experienced, and typically ends surgeries early. This can be taken into account during dynamic rescheduling.

The room state determination system can also be configured to automatically include timestamp information into an electronic medical record (EMR) data structure in relation to the procedure, such that the estimated duration of the procedure can be tracked, along with other ancillary information, such as how long turnover (and potentially sterilization) was between other procedures (e.g., to avoid the risk of contagion spreading, a major issue for hospitals in respect of antibiotic resistance).

In a further variation, the room state determination system can also have improved machine vision capabilities configured to estimate a surgical process state of a procedure, which can be used as part of the scheduling analysis or appending into the EMR. For example, for a surgery that requires laparoscopic tools, there may be individual states, such as preparation of the surgical area, the initial incision, the placement and insertion of the minimally invasive tools, the main surgery itself, and then finally the withdrawal of the tools and the subsequent surgical area closure.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 is a system architecture diagram showing an example practical implementation of a system for room state determination using machine vision, according to some embodiments.

FIG. 2A is a more detailed block schematic of the system of FIG. 1, according to some embodiments.

FIG. 2B is an example model pipeline, according to some embodiments.

FIG. 2C is a table that illustrates the performance of the validation set, according to different variations of the model architecture, according to some embodiments.

FIG. 2D is a graph showing receiver operating characteristic (ROC Curves) for a XGB Classifier, according to some embodiments.

FIG. 2E is a confusion matrix showing the predicted class against the true class, according to some embodiments.

FIG. 2F is a bar graph showing a class prediction error for the XGBClassifier, according to some embodiments.

FIG. 2G is a performance matrix, according to some embodiments.

FIG. 2H is a plot of the actual labels against predicted labels, according to some embodiments.

FIG. 2I shows an example camera feed screenshot.

FIG. 2J shows a Full Validation Set Confusion Matrix.

FIG. 2K shows a Partial Validation Set Confusion Matrix.

FIG. 3A is a chart showing a table of model performance across various permutations, according to some embodiments.

FIG. 3B is a graph showing technical issues relating to flickering from model confidence in an example scenario.

FIG. 4A, FIG. 4B, and FIG. 4C are heatmap diagrams showing flickering results based on confidence, according to some embodiments.

FIG. 5A is a plot that shows turnover time for cases delay or start early than 8 hours.

FIG. 5B is a plot that shows turnover time for cases started within 8 hours from scheduled time.

FIG. 6 is an example computing system diagram showing an example practical computing system for implementing the system for room state determination using machine vision, according to some embodiments.

### DETAILED DESCRIPTION

A computer implemented approach using machine vision machine learning model architectures processing a stream of digital images obtained by physical sensors, such as cameras or recording equipment is proposed. In particular, machine learning approaches are proposed that are adapted to operate in a high-volume, resource (processing resources and processing time) constrained environment.

As described herein, a specialized computer system is proposed that includes in-room video recording devices, such as cameras that generate a local stream that is processed to identify key transition milestones in terms of room state or procedure state, or both, which are stored as metadata tags that are time-synchronized to coordinate with other data streams and for time synchronized machine learning, where a machine learning model is configured to output to process machine vision inputs to generate predictions of the key transition milestones. An anti-jitter smoothing function may be applied to avoid undesirable state flickering at the decision boundaries associated with the different states.

The cameras or recording equipment operate in concert with a controller backend computing system that processes recordings made in one or a plurality of physical facilities, such as operating theatres, providing a healthcare “black box” recorder. A challenge with working with these recordings is that they are captured in a raw format and are both bandwidth and storage intensive.

For room state determination, an initial processing can be conducted locally using local computing resources at an earlier stage in the processing pipeline to obtain real or near-real time room state estimations, and the same video along with other recording data can be provided to a central server with more computing resources that is configured for generating predictive outputs relating to the procedure, such as predictively identifying errors, adherence to steps of a surgical safety checklist, among others.

Separating out the machine learning computations using separate models allows for a faster model for a faster response for the room state determination, and a slower model for a slower response for other types of cross-domain predictions or modifications, such as de-identification. The room state determinations can also be provided as a parallel input into the central server, the room state determinations usable as an additional signal for predictively identifying errors, adherence to steps of a surgical safety checklist, etc. In this example variation, the faster response for the room state determination is provided in parallel with the original inputs so that the slower machine learning model obtains the benefit of the pre-processing done locally for the room state determination.

The rooms are not necessarily surgical operating theaters but can include various types of rooms that are in use by professionals, such as dentists, dermatologists, among others, or non-operating rooms in a hospital or clinic, such as a trauma room.

In experimental testing, a number of different architectures were tested, as well as different computational optimization approaches.

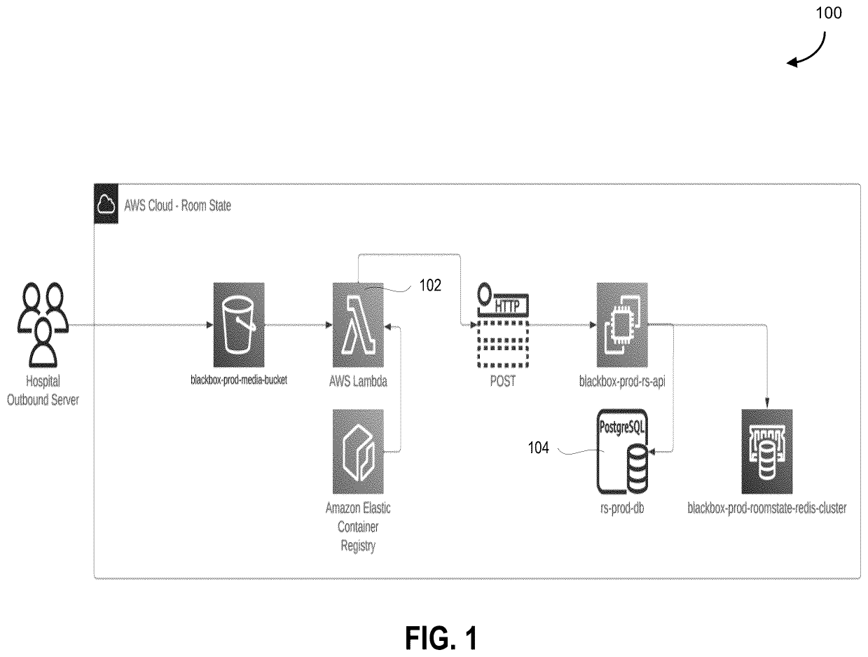

FIG. 1 is a system architecture diagram 100 showing an example practical implementation of a system for room state determination using machine vision, according to some embodiments.

Images are captured using are existing operating room cameras. For example, every 60 seconds, a frame is sent to an AWS S3 instance in an encrypted state for data security. Other process parameters are possible, but these are provided in an example.

Examples of room recording can include a stream of MP4 files that are provided on a continuous basis. These MP4 files include an audio track, a video track, and an associated metadata track that can be appended with data from sensors, equipment, etc.

For example, in a typical operating room, there can be 10 cameras, recording devices, etc. These create a stream of approximately 3 MB/s of recordings that are provided to the AWS S3 instance.

When images arrive in the AWS S3, this initiates the trigger for the AWS Lambda 102 function. The room state generated outputs can be stored on a database 104, which include specific transition milestones metadata tags that are time synchronized in one embodiment, or in another embodiment (e.g., at t=5 s, idle->case; at t=8281 s, case->turnover, at t=11049 s, turnover->case), or a stream of recorded states (t=8279 s, idle, t=8280 s, idle, t=8281 s, case, t=8282 s, case).

In some embodiments, a stream of recorded states is first generated, and then the stream of recorded states is pre-processed into transition timestamps and stored as a data object such as an array or a linked list having multiple time-synchronized data objects corresponding to each transition that can be stored as a record for the room or as a snippet into the corresponding case records, such as in an EMR to provide a more accurate estimation of how long a procedure took, which can be used to then compare actual time elapsed against allotted timeframes to provide more accurate schedule planning.

The lambda function 102 completes a number of tasks: 1) decryption, 2) DL inference, and 3) send inference result to Room State servers.

The reasons AWS Lambda 102 is used for this step in the data flow are as follows: 1) the serverless applications offers high scalability and request concurrency support, 2) relieves compute stress of API servers, and 3) economically viable relative to alternative methods such as AWS SageMaker™.

It is advantageous to be able to utilize processed outputs from the recordings to be able to assist with logistical decision making.

Accordingly, a computer implemented, machine learning approach is proposed that processes the recordings in real or near-real time such that logistical decision making can be supported through providing informational outputs on a user interface, or in some embodiments, logistical decision making is automated through invocation of secondary workflow processes that operate to re-arrange scheduling and bookings, such as room reservations and corresponding equipment setup in an attempt to optimize a particular outcome, such as an overall reduction in delayed cases, an overall reduction in when a last case is scheduled, etc.

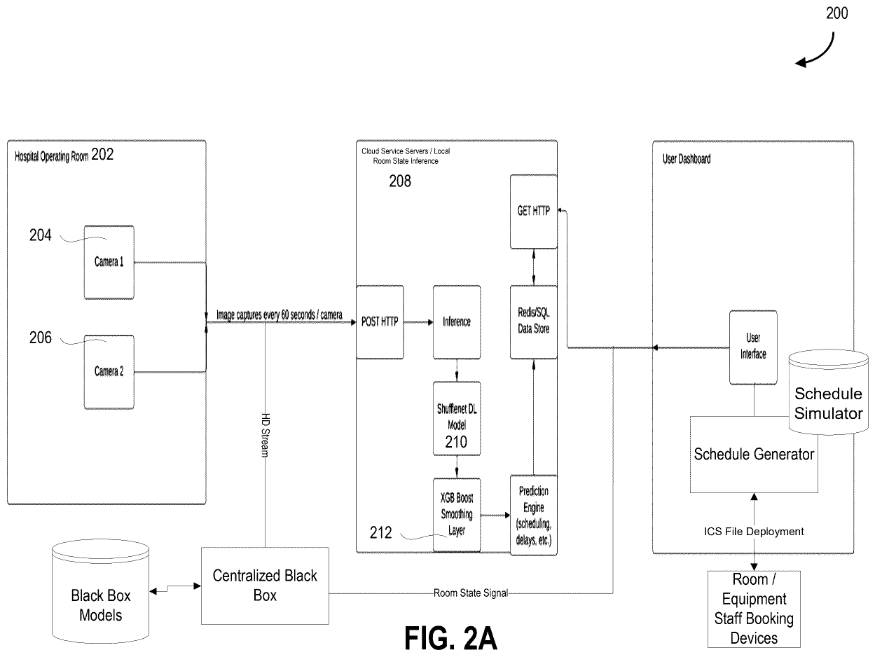

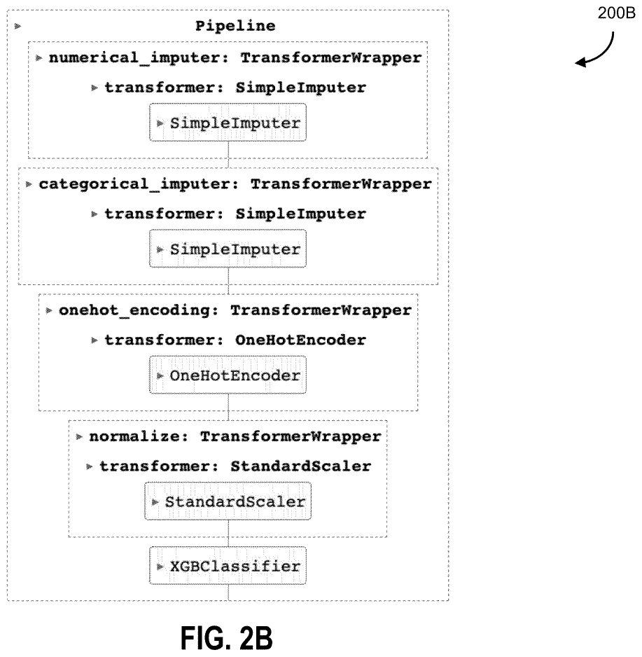

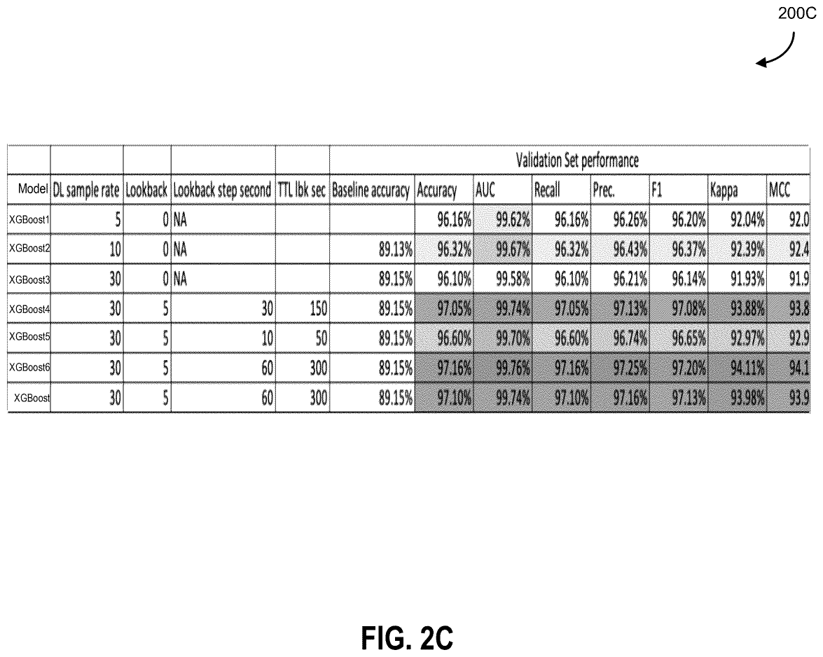

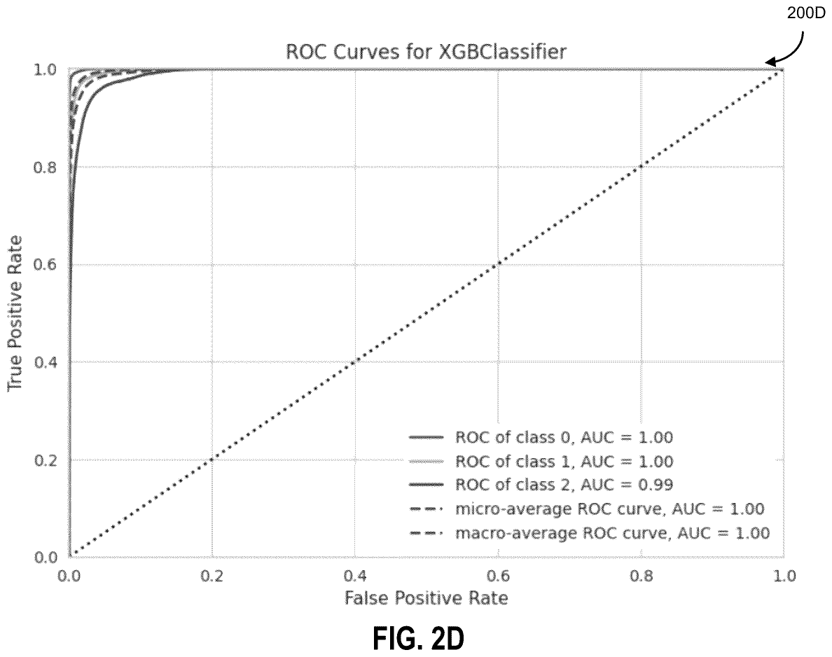

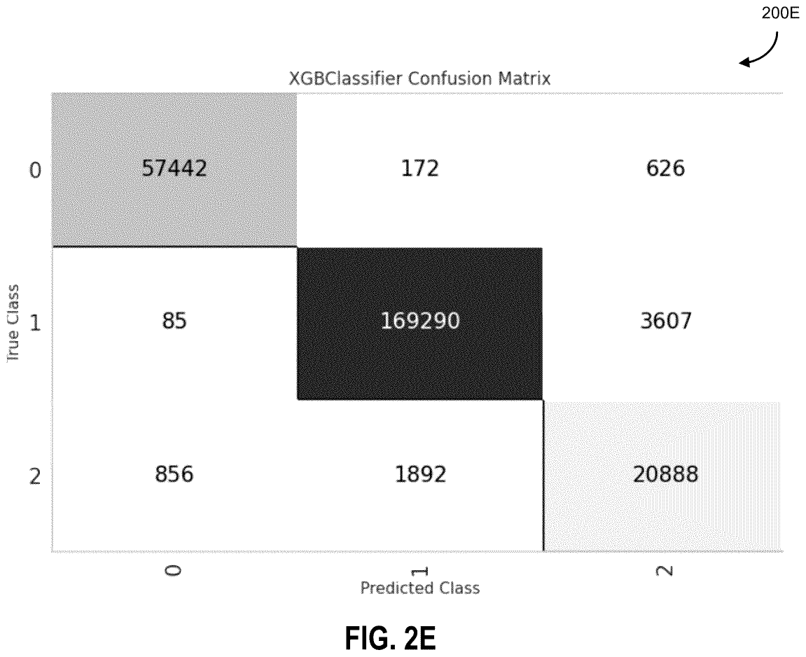

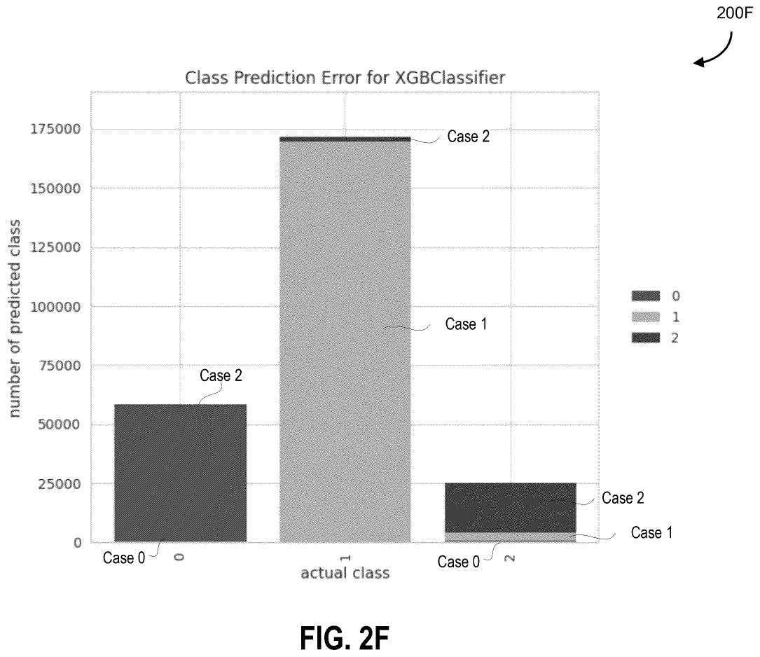

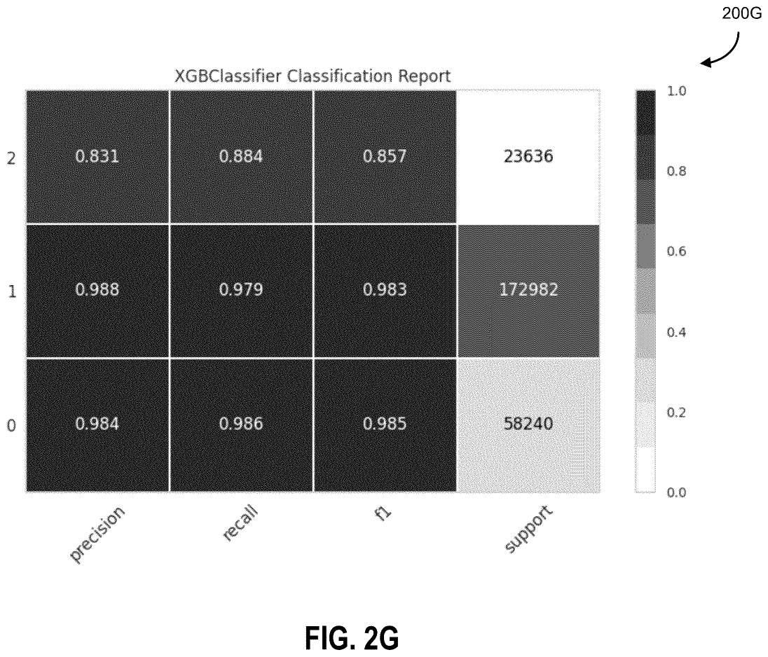

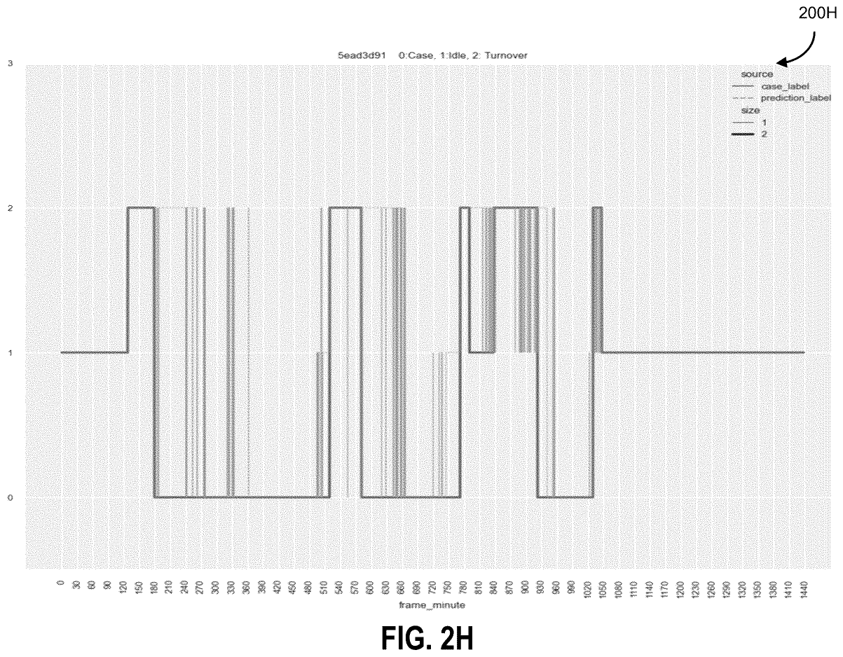

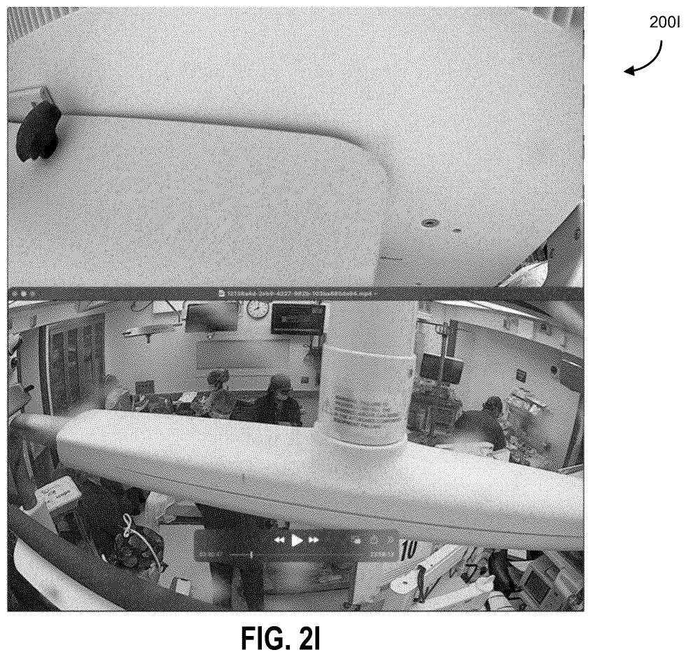

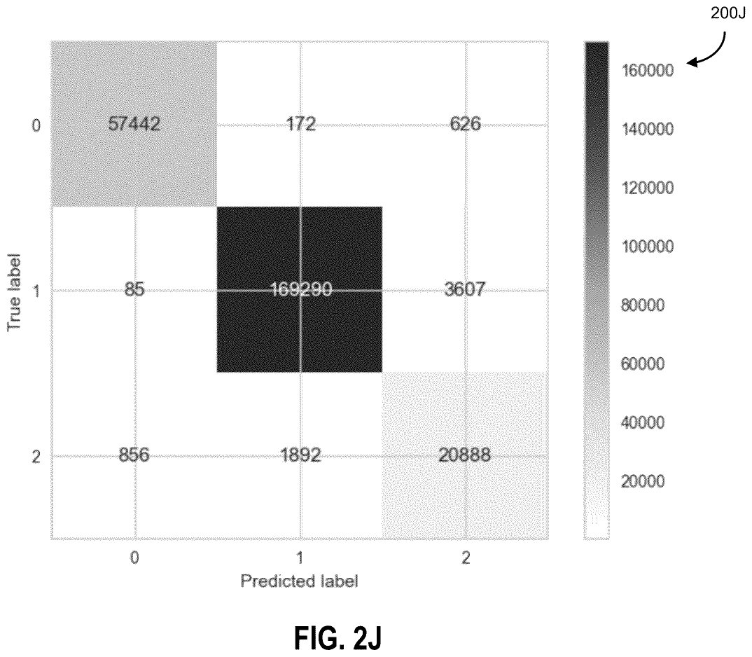

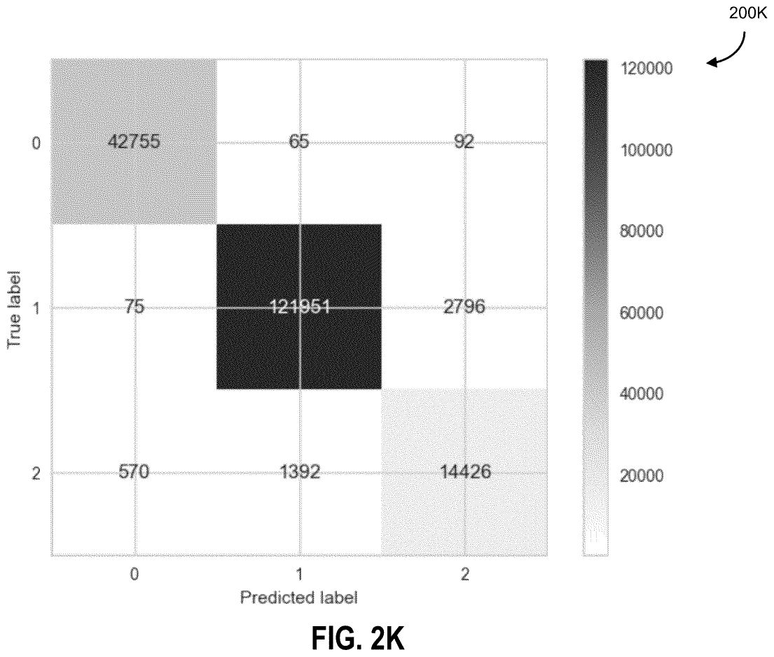

FIG. 2 is a more detailed block schematic 200 of the system of FIG. 1, according to some embodiments.

A hospital operating room 202 has the various cameras 204 and 206 working in concert, which interoperate with the cloud service servers using application programming interfaces, such as POST/GET HTTP interfaces, among others. As shown in FIG. 2, each room can have at least two cameras, for example wall mounted cameras, and the images of both cameras can be concatenated for a combined assessment based on interpretation of the combined feed, which helps avoid issues with occlusion where the field of view is blocked by people, equipment, or instruments.

Ideally, the cameras 204 and 206 should have different fields of view and provide images from different vantage points. Additional camera feeds are possible in a variant embodiment, which also includes using a camera feed whose field of view is directed towards a surgical field or an internal camera (such as cameras used for laparoscopic procedures) that is internal to the individual during the procedure. The benefit of having the camera directed towards the surgical field or the internal camera is that those camera feeds can be used for more specific room state determinations, such as intermediate determinations of what step of a procedure or a case to provide more accurate information points in respect of room state estimation.

In this example, a room state can principally have the major states of idle, case, and turnover, but the state “case” may have sub-states associated with it, which are the steps of the surgical procedure. The sub-states can be used for a more accurate downstream prediction of when a next transition will occur (e.g., a case is nearing completion when the sub-state is the closure of the surgical incision area), and the sub-state can then be utilized for a more accurate room state prediction for the optimizer.

While the cameras that are being used for room state can be the same cameras that are being used for a surgical black box, the cameras do not necessarily need to be the same. In some situations, specialized cameras can be used for room state determination, the images and feeds are used for the room state estimation and then discarded.

The camera feeds are provided to a local compute or, in some embodiments, a cloud computing server 208 that is configured for fast inference.

An inference classifier is proposed that is configured for applying transfer learning technique on machine learning architectures. In a practical implementation, the approach tested utilized a PyTorch Lightning framework and evaluated below architectures:
Resnet 18
Resnet 50
Resnet 152
Shufflenet
SwAV

The training strategy included first training the additional classifier layer for 10 epochs. For 11 to 25 epochs, the approach included relaxing all pre-trained model weights to further boost model performance.

It was found that Shufflenet and Resnet 50 have similar performance, and that Resnet was slightly better than Shufflenet. The best model selection is based on validation loss during the training process. While a Shufflenet DL model 210 is shown in FIG. 2, in other variations, a Resnet model is possible instead.

For the smoothing layer 212, ensemble approaches are proposed for boosting ML model performance. The smoothing layer 212 helps to further improve the DL model results. Due to the nature of video, the system can treat the video frames as time series data. A frame label at time t can be inferred by previous frames, e.g., frame t−1.

Based on this observation, the system then is configured to apply data engineering techniques to generate a rich informative model input from the DL model output. In the first model version, Applicants attempted to append the previous x frames as predictive features.

A second set of mp4 files and corresponding annotations files were used for sampling as well. An updated script was used in Workflow API, and the frames were inferred by the trained DL model 210. The annotations are set as the ground truth. Applicants chose logistic regression, Xgboost, Catboost and LightGradientBosst models for experimentation.

As described above, each surgical room 202 has at least 2 room cameras 204 and 206 in the experimental setup. Therefore, the system can use two or more camera's information in an effort to predict the current room state.

Example tabular format shows at below at Table 1:

| Frame Time | Feature from feed 1 | Feature from feed 2 |
|---|---|---|
| T0 | DL model output at t = T0 | DL model output at t = T0 |
| T1 | DL model output at t = T1 | DL model output at t = T1 |

### Engineered Feature Notes:

The time related features are important for scheduling, and thus following features are created: Weekday, from 0 to 6, Hour of the date, from 0 to 23, Minute of the hour, Month, day of the year/month. Experiments were conducted based on 2 months of data.

Time in stage: this feature is created using annotation data due to DL modeling output not being 100% accurate. In production usage, the approach may utilize data based on previous Xgboost model predictions.

The model can be used for real time operation, and in real time operation, the analysis can only utilize backward time series setting. However, in certain scenarios where the real-time operation aspect is not needed (e.g., a room state can be generated with a slight delay or entirely asynchronously), forward looking time series settings can also be used to improve accuracy. During real time operation, when cases begin taking longer than expected compared to schedule or a predicted time, the schedule generator engine can be configured for automatically extending scheduled time on a reservation system to match the actual allotted time based on the room state estimations generated by the system.

The Model Training Script can have Different Setting Parameters:

DL sampling rate: simulate the upstream DL model inference rate. Default value is 5 seconds.

Lookback number: number of frames from current time step are used for prediction.

Lookback sampling rate: default 5 second.

The ML training script that can be configured to utilize the Pycaret package. The model structure is illustrated in FIG. 2B, at the example pipeline illustrated at 200B.

During experimental performance review, due to training instance memory limitations, Xgboost was used for 1st iteration. Catboost was only trained with best hyper parameter setting of Xgboost. The baseline (DL output) accuracy was found to be around 90%.

A script was established to load the trained models and to run inference using the validation dataset. All misclassified frames were logged with file path, true label, predicted label, and raw model outputs for manual check. The validation result shows that most misclassified frames are below to following types, which are hard to distinguish.
The room camera is covered by either instrument or door.
The transition between stage to stage, e.g., Idle to Turnover or Case to Turnover.
Mislabeled annotation, e.g., annotated as Idle but observed worker clean surgery room.

FIG. 2C is a table 200C that illustrates the performance of the validation set, according to different variations of the model architecture, according to some embodiments.

FIG. 2D is a graph showing receiver operating characteristic (ROC Curves) for a XGB Classifier, according to some embodiments. In FIG. 2D, the graph 200D is a visual representation of the model performance across thresholds. As shown in FIG. 2D, a graph is shown charting the true positive rate against the false positive rate, and as shown the model performance is satisfactory. FIG. 2D shows plots for Xgboost 6, where the classes correspond to 0: Case, 1: Idle, 2: Turnover.

FIG. 2E is a confusion matrix showing the predicted class against the true class, according to some embodiments. The confusion matrix 200E is from experimentations with the XGBoost 6 model.

FIG. 2F is a bar graph showing a class prediction error for the XGBClassifier, according to some embodiments. Bar graph 200F plots the actual class against the number of predicted classes, and similarly, the classes correspond to 0: Case, 1: Idle, 2: Turnover.

FIG. 2G is a performance matrix, according to some embodiments. The performance matrix of 200G shows the performance of the XGBoost 6 model as experimentally validated.

Overall, it was found experimentally that the Xgboost6 model performance is good, but there may be misclassified frames that need to be examined to determine if the model has unexpected behavior.

There are two types misclassified conditions, including: the worst performed module arose due to cameras being covered by instruments. The time ordered labels and predictions are plotted in FIG. 2H with a camera feed sample in FIG. 2I.

FIG. 2H is a plot 200H of the actual labels against predicted labels, according to some embodiments. FIG. 2I shows an example camera feed screenshot 200I.

Other misclassified frames were determined due to mislabeling and can be categorized as the following:
label idle, predict turnover: the model is sensitive to people in the room, and if a worker is not just sitting and waiting, the model will predict cleaning.
label case, predict turnover: this issue occurred in early stages of a case state. If a patient is covered by instrument or there is a poor view for the camera, model will inadvertently predict turnover.
label case/turnover, predict idle: if the frame shows no people in the room, model may have a tendency to predict idle.

In an embodiment, the approach can include excluding the video tracks from devices which accuracy is less than 80%, and there may be a performance improvement relative to a full validation set. The detailed metric table and confusion matrixes are provided at FIG. 2J and FIG. 2K. FIG. 2J shows a Full Validation Set Confusion Matrix 200J, and FIG. 2K shows a Partial Validation Set Confusion Matrix 200K.

Based on the results, all types of metrics were improved after excluding those feeds. Based on the improvements, when utilizing the model for predict the room state, the system can be configured to be biased to automatically use higher accuracy feeds first. Note the table should be updated and monitored frequently in case of data drifting.

A table is provided below in respect of the data outputs, showing accuracy using different validation sets.

| | | Macro | Macro | Macro | Macro |
|---|---|---|---|---|---|
| | Accuracy | Accuracy | Prec. | Recall | F1 |
| Full | 97.2% | 95.0% | 93.4% | 95.0% | 94.2% |
| Validation | | | | | |
| Set | | | | | |
| Partial | 97.3% | 95.1% | 93.6% | 95.1% | 94.3% |
| Validation | | | | | |
| Set | | | | | |

The room states are based on machine learning outputs based on processing of raw surgical recordings obtained by the hospital operating room. Because there can be a large number of recordings being processed at any given time, bandwidth and processing constraint considerations are important. Specific approaches are also proposed in various embodiments to assist in reducing computing burden in an attempt to provide outputs within a desired timeframe, even at a cost of accuracy.

The model for inference is described in an example below, and it is operated at the cloud service server level. There is an inference engine, that works with deep learning models and a smoothing model layer that provides inputs that are ultimately utilized by the prediction engine for generating scheduling, identifying delays, among others.

The first layer is a deep learning model that processes the decrypted image.

The decrypted images are received as individual frames, and individual frames are processed at the first layer. The first layer takes as input features specific pixels of the individual frames for analysis. The first layer then outputs a serious of probabilities describing the most probable state for the current room that is then provided to the second layer.

The second layer is a layer including gradient boosting trees, providing ‘smoothing’ model (XGBoost) that takes as input current and past predictions to better inform the prediction of the current state. The reasons for the gradient boosting trees is that there is correlation as between different states. The system is configured to “look backwards” at X states, and more recent states are applied higher weightings to bias the system towards the more recent states. Effectively, the current state has predictive power over the next state.

In a practical example, the processing is conducted at a frame-by-frame level. For example, since states happen in sequence (e.g., Case for 3 hrs, then turnover for 30 mins, etc.), if the current frame is Case, this increases the probability that the next frame will also be case.

The final layer is a rules-based anti-flickering model that increases the barrier to changing the state, and therefore decreases the frequency of flicker states.

An example of a flicker state can occur as a result of a person entering a room in the middle of the night. An operating room is idle most nights.

However, if someone briefly enters the room and starts looking around for say, a forgotten laptop, then this might lead the model to mistakenly assess that there is a turnover state commencing.

The system avoids flicker states in such scenarios by increasing the threshold for allowing a state change. Specifically, for the model to report a changed state, it needs to either be the case that a) the model has a very high confidence score for this state (>90%) or b) this new state, despite the model yielding a low confidence score, has been observed for 5+ minutes).

For deep learning inference, transfer learning is proposed using the Shufflenet CNN. The model takes in a resized image and as output predicts the image to encapsulate one of 3 states: Case, Turnover, or Idle. The reason there is a resizing the image to 224×224 is because this is the standard Shufflenet size. The reason three output classes were chosen is because they sufficiently capture the user value, while also allowing for sufficiently high performance.

During training, Applicants evaluated a variety of models (resnet, inception net, mobilenet, etc.). Another reason for selecting the Shufflenet model is because it is a relatively small model thus allowing for faster inference. This is a critical requirement for the use case as Applicants seek to run inference in real time at scale.

A smoothing model is provided, because consecutive frames are not independent. In other words, the probability that the current frame is a case state given that the prior frame is a case state is different than the probability that the current frame is at a particular case state. Hence, the approach leverages these dependencies to create better predictions by using a smoothing model. Similar to the deep learning stage, the approach is adapted for training a variety of models (e.g., XGBoost, LightGBM, etc.), and XGBoost was found to yield the highest performance.

A user dashboard is provided that includes a user interface where a user is able to view schedules (and associated delays), select variations on schedules based on existing and expected delays (e.g., based on an identification of one or more aspects to optimize, such as reducing overall overtime costs where the facility needs to be kept open). The dashboard can include both the current room state for each operating room, but also a generated prediction of future room states based on a current schedule or a simulated schedule, automatically generated by the system periodically or as triggered. The simulated schedule can include proposed re-scheduling and re-assignment of resources, and in some embodiments, the system automatically manages the schedule by dynamically operating the schedule and only “locking in” a schedule at a short duration into the future (e.g., 2-3 hours), so that the system can continually seek to optimize an objective function. In this example, the dashboard shows the existing room states, as well as predictions in relation to estimated overtime hours/cost and an expected end time to all scheduled procedures based on the simulated schedules. In some embodiments, when a simulated schedule is about to be locked in, a human reviewer is notified to provide an approval indication on the dashboard through an interactive graphical control element before resource assignment data messages are generated and sent.

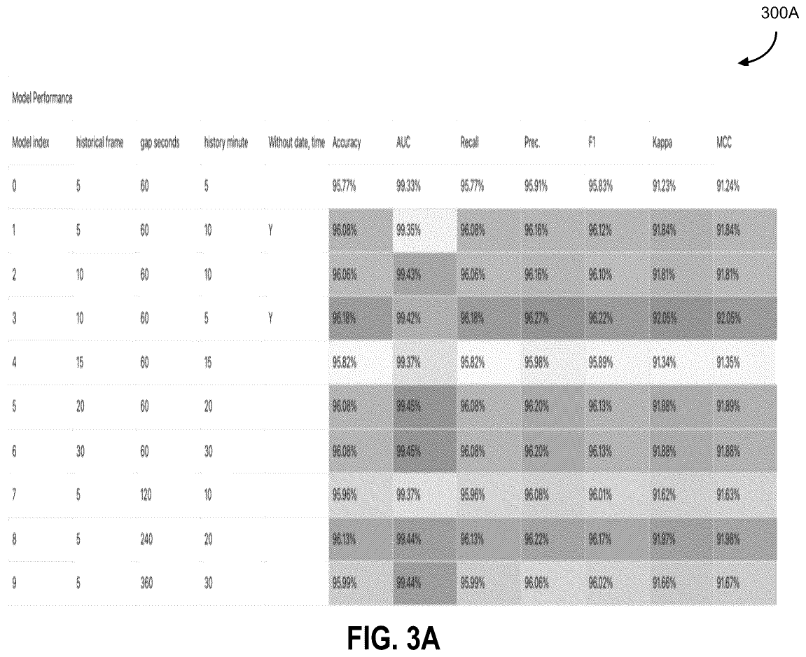

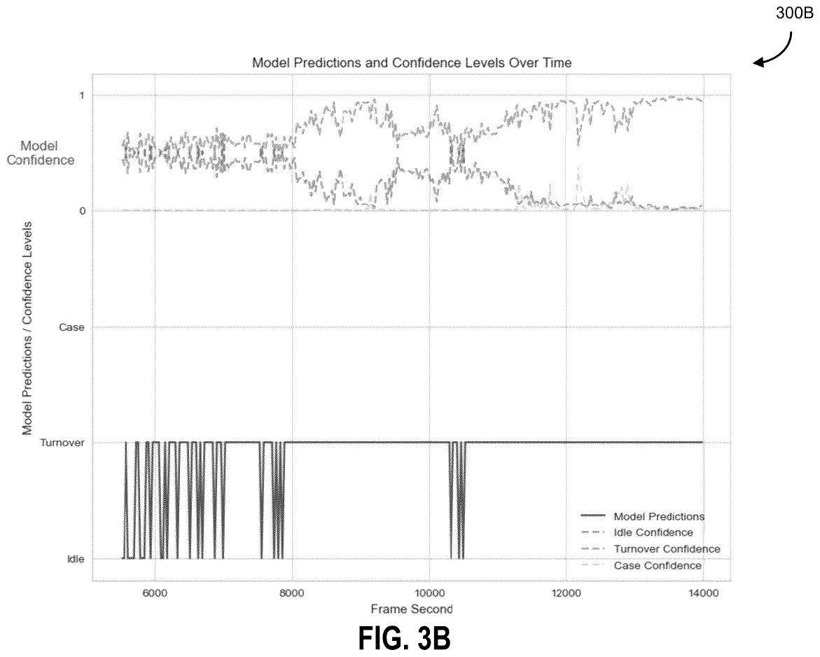

Similarly, feature selection consisted primarily of optimizing on multiple axes: inference from how many frames in the past are observed, and the amount of time between frames. There are also included features such as the time of the day (since the probability that the state is case is greater at 3 μm than it is at 1 am. For example, see the iteration of permutations in FIG. 3A.

Because the optimal delta between sequential frame inference that is inserted into the smoothing model is 60 seconds, the system receives a new image from each camera every 60 seconds. The other hyperparameter that was selected was 5 historical frames such there would be an established “look back” 5 minutes in the past. While a smoothing model can be used that looks both forward and backwards when making a prediction for a given frame, since there is a desire for designing this pipeline to support a real-time application, the system is configured to only allow it to look backwards. Additionally, there are multiple cameras in the operating room.

Since for any given frame a given camera could be blocked or obstructed, in the smoothing model, Applicants leverage the deep learning inference output from 2 cameras. Additional cameras can also be utilized. The deep learning model performs inference on individual frames. The smoothing model leverages multiple cameras working in concert, and can assist with obstructions, etc.

The final stage of the inference pipeline is the flickering reduction model. A flickering reduction model is utilized is because even despite the layers above, since the output of this pipeline is customer facing, it is essential that the approach limits data noise.

As described above, while the room state model output after XGBoost smoothing is satisfactory, there is room for improvement. Specifically, it is a technical issue that the current model output encounters a technical issue relating to flickering (switching from one state to another state rapidly or jumping back and forth between two states) as the determination can be impacted by unpredictable events, such as a person walking into a room to retrieve a forgotten computer, a worker taking a break from cleaning, among others. In this example, one individual in the operating room would likely yield a low confidence score, and hence they would have to be actively looking around the operating room for over 5 minutes, a low probability scenario.

An example graph is shown at FIG. 3B, showing technical issues relating to flickering in a graph showing model confidence in an example scenario. It is observed that flickering usually happens around the Turnover stage, when there are personnel coming in and out operation rooms but there is not actual setup segment or cleanup segments. It could be very early in the morning long before all scheduled cases or very late in the night long after all cases. Flickering also could happen in the early stage of an actual setup segment.

The root cause is that the model's output probabilities for turnover and idle are similar, indicating low confidence in class differentiation. The use of the argmax function can lead to prediction oscillation or instability, as the predicted class may switch back and forth, or ‘flicker’, between the two classes over successive iterations. Another flickering could happen for very confident cases or idle predictions. It is a rare scenario, but sometimes the patient is pushed out of OR room or suddenly the system detects no people during non-idle segments for the moment. While such prediction is valid, the outcome is still suboptimal from a state determination perspective, and the system is configured to minimize such flickering.

The proposed technical solution to address the technical issues relating to flickering include using a proposed waiting buffer, which only changes to a new state if a consecutive n sequence of new class prediction has been detected. If the current prediction as output by the XGBoost smoothing model differs from the previous confirm state, the flickering reduction model will only allow a shift if a) the model is highly confident in this prediction or b) the model has predicted the same new state for 5 predictions in a row despite being low confidence.

An improvement on the improvement on the waiting buffer approach can be established where a delay in prediction is undesired. In the improved waiting buffer approach, if current model output has high confidence (e.g., greater than a threshold), the system can be configured to state sooner to minimize delay.

From a specific implementation perspective, this approach can use two buffers, a first buffer: record number of consistent diverging predictions after previous stable prediction, as well as a second, high confidence buffer: record number of high confident diverging predictions after previous stable prediction. The second buffer has a smaller size than the first buffer size and when either buffer is met, the state changes. This combined buffer approach provides a useful mechanism to avoid undesirable state changes at the transition points.

The flickering reduction model takes as input the current prediction and the past few, as well as the current state.

Choosing the optimal thresholds here was an iterative exercise. Implementing the waiting buffer to smooth flickering has benefits and drawbacks. On one hand, the approach addresses the technical problem and reduce the impact of flickering segments significantly. On the other hand, for a real time product, if a case starts and the system waits several minutes to reflect the change, it is also not acceptable.

Metrics considered included observing the average flickering segment per day, which measures how many additional segments were predicted compared to ground truth labels, where lower is better; observing the turnover segment accuracy difference [original-smoothed], which measures difference in turnover segment accuracy between original and smoothed output. Turnover segment accuracy is defined as the # of turnover segment picked up by model output/total # of ground truth turnover segments, where >0 means better, <0 means worse.

Since this is a difference, then original XGBoost has a baseline accuracy difference of 0. The approach included missed segment duration in minutes to capture any additional missing segments after applying smoothing. The missed segment duration list is the set difference between original and smoothed. It means a smoothed prediction missed this segment of this duration while the original prediction did not, but it does not imply smoothed prediction will always miss such segment.

Another metric under consideration was the point Accuracy difference (smoothed−original), which measures accuracy difference against ground truth labels across all timestamps, and >0 means better, <0 means worse, where the original XGBoost has a baseline accuracy difference of 0.

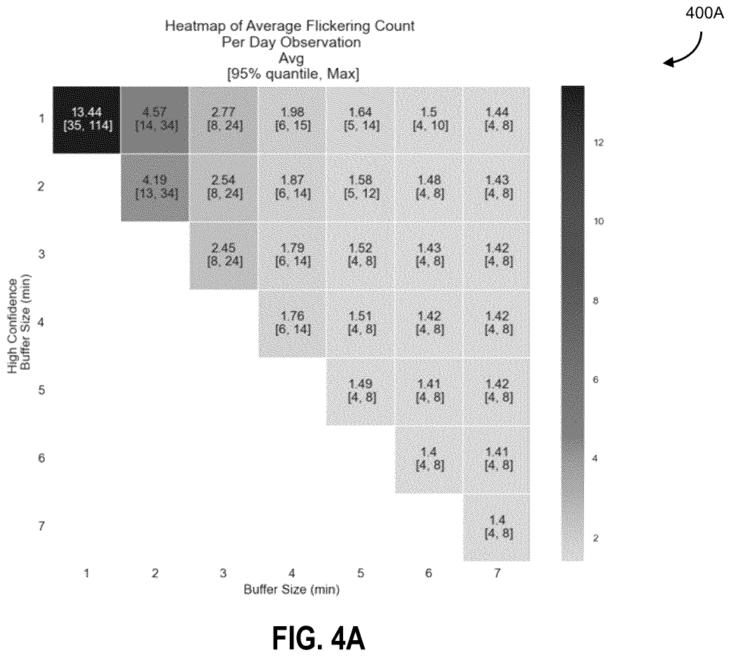

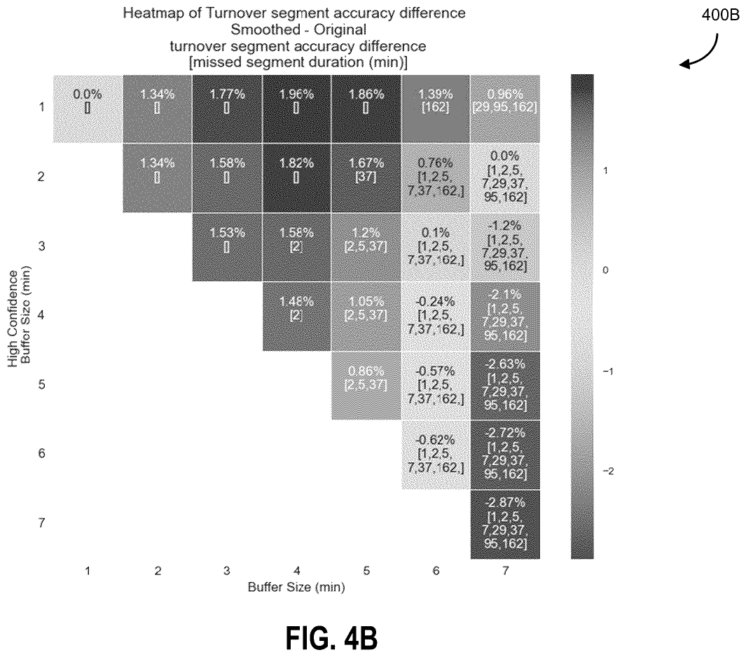

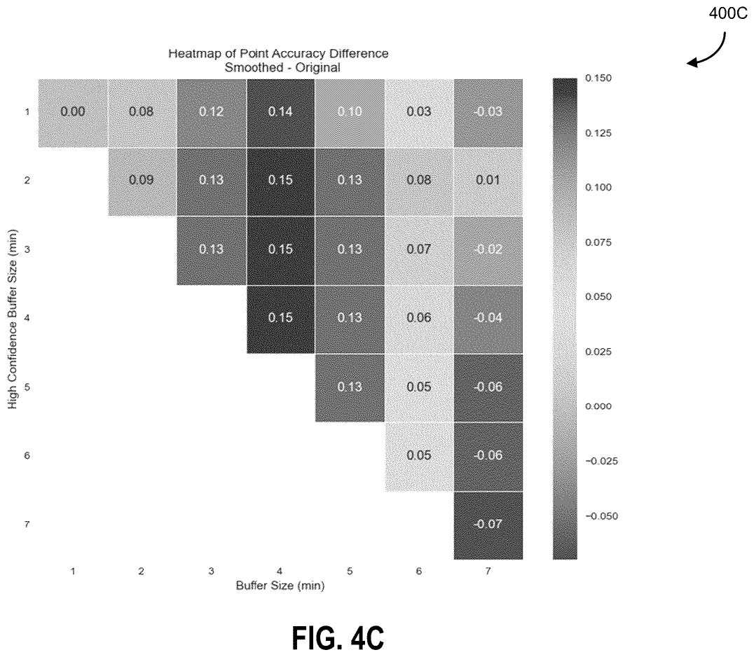

The image in FIG. 4A, FIG. 4B, and FIG. 4C captures the results from various configurations. The plots were generated over 484 day observations. In the top left corner of each plot where buffer=1, the original XGBoost output (baseline) is shown. In analysis, it was found that adding a buffer reduces flickering greatly, but there was also observed a strong diminishing of return once the buffer size reached 6 or 7. The same results were observed on turnover segment accuracy and point accuracy, there was observed an improvement around buffer size of 4 and then performance actually decreases as one increases the buffer size, and as experimentally validated, the latter two plots narrow the search space to buffer size of 3-5. Ultimately, from a practical application perspective, it was found that a buffer size of 5 for the first buffer, and a smaller buffer size of 2 or 1 for the high confidence buffer size worked best.

For the high confidence buffer size, the buffer size of 2 was useful for a room state output that was based on a whole day chunk. The reason for this was that the system can correct previous predictions locally and give corrected outputs. Accordingly, a high confidence buffer size of 2 and buffer size of 5 given the rationale in the previous analysis.

For the high confidence buffer size, the buffer size of 1 was useful for a room state output that was being used to output room state output in real time per minute. Due to the nature of prediction accessing and storing patterns (via Redis), it is very difficult to modify prior inference, so in this situation, a high confidence buffer size of 1 was selected.

Given the described flow of producing a prediction state for the current room status, the next objective in this product is to provide a prediction engine component that is configured to communicate to a user (1) what this status is, (2) how this status aligns with the hospital's schedule and (3) predicting when the remaining cases will start and finish that day.

The room state output can be provided in the form of a decision support interface, such as a portal for an administrator who is able to view existing room states and downstream estimated room states or expected delay metrics based on the room states. The administrator, in some embodiments, may also provide corrections to the room states for future re-training of the model.

The first step in being able to communicate steps 2) and 3) is matching the hospital schedule to the observed cases from the model. This involves sequentially ordering the cases in the schedule and those that were observed, and then matching them in sequence if they satisfy a series of constraints (e.g., actual cannot happen too far in advance of the schedule, actual case duration cannot be too much shorter than scheduled case duration, etc.).

Finally, once matching is complete, there may be a series of cases that still need to happen. From this, the system can be configured to create a list of states to come (i.e., there is turnover before and after each case, plus the system needs to predict the duration of the cases themselves). There also needs to be predictions of the time remaining in the current state.

A room state matching engine is described below. It is non-trivial to match room state observed segments to scheduled cases. In some embodiments, a preliminary pre-processing occurs before passing the observed segments into the actual matcher. If the time gap between the end of one case and the start of the next is 480 seconds (8 minutes) or less, the two cases will be merged into one. This merging reduces the number of candidates segments for combination match to improve matching speed. However, there can be more complex and robust merging possibilities in combination matching step after this preliminary matching. If an observed case segment has a duration of 300 seconds (5 minutes) or less, it will be converted to a turnover segment instead.

The matcher engines first tries to match using wheels_in and wheels_out time only and match the remaining observed segments using combination matching. In some cases, the system can receive real-time updates for the wheels_in and wheels_out times for scheduled cases. These updates are used as the ground truth for constructing the matched segments.

When both wheels_in and wheels_out times are available for a scheduled case, the matcher engine uses these times to identify the closest corresponding observed segments. Specifically, it matches the wheels_in time to the start of the closest observed segment and the wheels_out time to the end of the closest observed segment.

If both times match the same observed segment, no merging is needed. However, if they match different observed segments, these segments are merged as one observed segment.

If the system only has wheels_in available, the system can use that specific timestamp to split observations into two parts: before wheels_in and after wheels_in, and then apply combination matching separately.

If the system only have wheels_out available, the system use that specific timestamp to split observations into two parts: before wheels_out and after wheels_out, and then apply combination matching separately.

For any remaining unmatched observed case segments, the system can be configured to attempt to automatically explore the possibilities of merging and omissions (potentially unscheduled cases) and choose the optimal combination with the lowest loss score. Loss score determination is described further below.

Observed case segments are merged greedily (via smallest gap first) to reduce total number under or equal to 8. This is to ensure efficient combination calculation since this algorithm scales exponentially in time.

### Possible Combinations:

Scheduled cases: A matching approach for scheduled cases can include match 1, 2, 3, . . . up to n scheduled cases to observed cases.

Observed Cases Omissions: If there are n scheduled cases and m observed cases where m>=n, the system generates combinations for observed cases from n to m, effectively omitting some observed case segments (treating them as unscheduled cases).

Observed Cases Merging: If there are n scheduled cases and m observed cases where m>n, the system generates combinations of merging m segments into n cases. There is a merging threshold of 2500 seconds (41.6 mins). If the gap between observed cases to be merged is larger than this threshold, the system can be configured to drop such combinations.

Loss score determination can include losses comes from two parts: duration mismatch and start time mismatch. The system can be configured with the assumption that the observed case should have a duration and a start time closest to those from the schedule case. Duration loss was the sMAPE loss between scheduled duration and actual duration, determined using a relation 2*abs (observed_duration−scheduled_duration)/(scheduled_duration+observed_duration).

The loss can be capped at 2, and the duration loss can be set at infinite (e.g., 10,000) if the observed duration is less than 16.7% of the scheduled duration, effectively dropping that combination. The start time loss was the time difference normalized by hour in log scale, and the loss is also capped at 2, which is capped at a 7-hour difference.

Given the knowledge of which states for which the system needs to predict durations for, a simulation is conducted to return a distribution of the start and end times of each state that remains.

Running simulations involves generating distributions for each state type.

The simulation can be conducted as follows:

Obtain the current time and the current state. Given distribution of given state, sample the conditional expectation on the remaining time of the state, given the elapsed time.

Given result from the above, append sampled duration to current time. For each remaining state, sample the duration given the state specific distribution. Each state's end time will be the ‘current time’+the sample duration. Each state will append the duration to the ‘current time’, thus providing timestamp distributions for the start and end of each state.

Repeat multiple (e.g., 1000) times, while storing in memory each state's start and end for each simulation run. Accordingly, an estimated duration, for example, can be applied to a particular state. This is useful as the room states will change and it is important from a dynamic optimization perspective.

A number of methods were attempted to produce the predicted timeline.

The reason this method was ultimately selected was because of a combination of factors including (a) a rich feature set that becomes possible and (b) accuracy. Some other methods attempted including using just the mean expected value for each state. However, the accuracy performance of this method did not justify the decreased feature set (i.e., no case start, end, and duration distributions).

To generate the distribution for room turnover, the system uses XGBoost to output the distribution parameters.

This involved a few steps, firstly identifying which distribution best models turnovers. This was found to be the Gamma Distribution. From there, Applicants identified the features that impact the expected duration of a turnover. Examples of such features include the specialty of the upcoming case, whether the prior and next case share the same specialty, the facility where the turnover is taking place, etc. Populating these features among others, Applicants then use the XGB model to output the parameters of the Gamma Distribution. This output then feeds the simulation process above. XGBoost was chosen because it had the best results when compared to similar models.

The scheduling model follows a very similar process to the turnover model though with a richer and expanded set of features. The scheduling model can be used, for example, for dynamic room assignments. Turnover definition is the time from first case wheels out timestamp to second case's wheels in timestamp. Note that other definitions are possible, such as a more complex definition like and case cleanup, but for this non-limiting illustrative example, the scheduling model focuses on the gap between 2 consecutive cases.

The approach can adopt same data cleaning processes were used in case modeling, including following components: correct negative schedule time and case time, clean up procedure specialty, find primary surgeon of each case, find 1st available procedure and apply BBP mapping.

Cleaned case data is prepared from the above, then the system can be configured to start matching cases to build and update a daily scheduled timeline data structure.

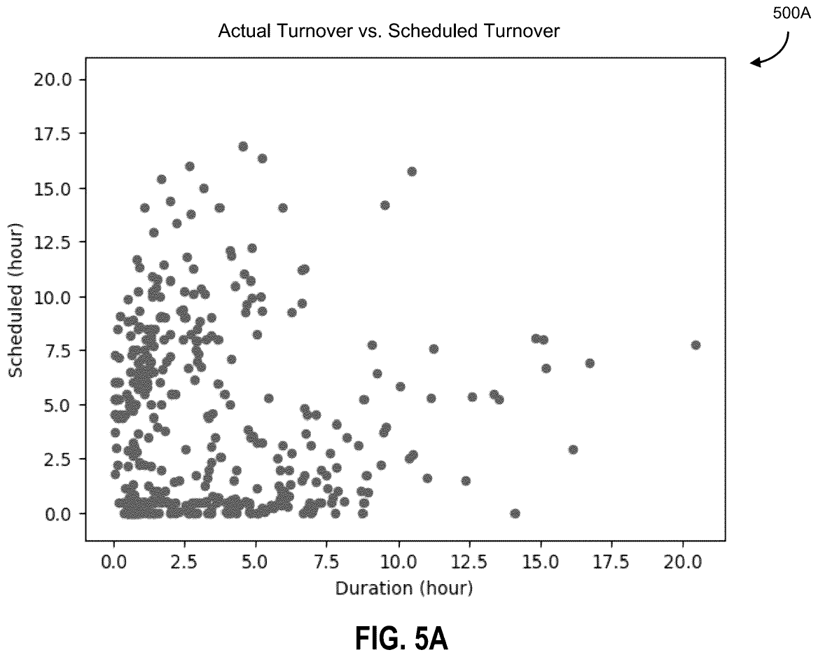

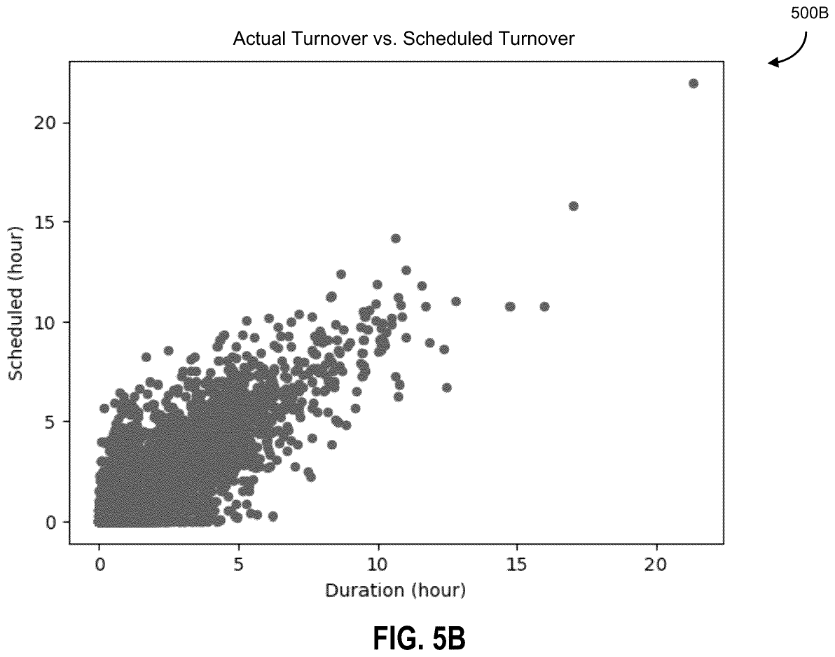

The following logic was used for matching: (1) Given a case (prior case), find all other cases scheduled in the same day in the same room, (2) Compute the time difference between (prior) case scheduled end time to other cases scheduled start time as scheduled turnover time, (3) Find case with minimum positive scheduled turnover time as post case. (4) Compute the actual turnover time based on prior case's wheels_out and post case's wheels_in. (5) Based on plots generated, FIG. 5A and FIG. 5B, from a practical perspective, the system was configured to exclude cases are delayed or started earlier than 8 hours as it was difficult to predict those extreme cases.

FIG. 5A is a plot 500A that shows turnover time for cases delay or start early than 8 hours. FIG. 5B is a plot 500B that shows turnover time for cases started within 8 hours from scheduled time.

From a feature engineering perspective, the turnover model features can include:

Schedule related features (Weekday of the day, Prior case scheduled in the morning, Post case scheduled in the morning, Are prior and post cases surgeon the same, Are prior and post cases specialty the same, Scheduled turnover time, Prior case scheduled case time, Post case scheduled case time, Prior case scheduled start time hour of the day, Post case scheduled start time hour of the day)

Time related features: Because this model can be used in real time, the system will have more information following the occurrence of an event. Note that, prior to those events happening, all those features should be unknown. These features include:
When time moves to prior case scheduled time or prior case started (Has prior case started early (or delayed)?, How much time the case started early or delay)
When time moves to prior case scheduled end or prior case ended (Is prior case delayed or ended early, How much time the delayed or ended early)
When time moves to prior case ended (the system can update scheduled turnover time from prior case wheels_out timestamp to a post case's scheduled_start timestamp)
When time moves to post case's scheduled_start timestamp, but the post case has not started yet, the system can determine that the post case is delayed.

Modelling results are shown in the below two tables.

The stage is referring to different event happened in the prior or post case. 1st stage is where the system only knows the cases schedule. The 2, 3, 4, 5 stages can be mapped to previous section 2-a to 2-d. Delay Schedule is the scheduled MAE for delayed cases only. Note this table is only for cases happened within 4 hours delay or start early.

| | | 1st | 2nd | 3rd | 4th | Delay | 5th |
|---|---|---|---|---|---|---|---|
| Facility | Schedule | Stage | Stage | Stage | Stage | Schedule | Stage |
| Loca- | 60.3 | 29.5 | 29.1 | 28.9 | 28.6 | 59.6 | 27.1 |
| tion 1 | | | | | | | |
| Loca- | 39.2 | 32.0 | 30.0 | 26.4 | 24.7 | 38.5 | 22.2 |
| tion 2 | | | | | | | |
| Loca- | 36.4 | 11.7 | 12.2 | 13.2 | 13.0 | 37.7 | 10.9 |
| tion 3 | | | | | | | |
| Loca- | 31.6 | 13.6 | 13.2 | 12.6 | 12.2 | 30.8 | 10.3 |
| tion 4 | | | | | | | |
| Loca- | 26.6 | 16.9 | 17.3 | 16.9 | 17.9 | 27.0 | 18.3 |
| tion 5 | | | | | | | |
| Loca- | 25.6 | 21.8 | 21.0 | 19.8 | 19.1 | 21.8 | 12.1 |
| tion 6 | | | | | | | |
| Loca- | 21.5 | 15.1 | 14.7 | 13.6 | 13.3 | 21.1 | 13.0 |
| tion 7 | | | | | | | |

The below table contains the modeling results if the configuration relaxes the 4 hours threshold to 6 and 8 hours.

| | Schedule | | | | | | |
|---|---|---|---|---|---|---|---|
| | 4 hr/6 hr/ | 1st | 2nd | 3rd | 4th | Delay | 5th |
| Facility | 8 hr | Stage | Stage | Stage | Stage | Schedule | Stage |
| Location 1 | 60/67/72 | 30/34/39 | 29/34/39 | 29/34/39 | 29/34/39 | 60/64/67 | 27/31/36 |
| Location 2 | 39/40/41 | 32/33/35 | 30/32/33 | 26/29/31 | 25/27/29 | 39/39/40 | 22/23/27 |
| Location 3 | 36/37/37 | 12/12/12 | 12/12/12 | 13/13/13 | 13/13/13 | 38/38/38 | 11/11/11 |
| Location 4 | 31/33/34 | 14/15/16 | 13/15/16 | 13/14/15 | 12/4/15 | 31/31/31 | 10/11/12 |
| Location 5 | 27/30/31 | 17/18/20 | 17/18/20 | 17/19/21 | 18/20/21 | 27/22/23 | 18/14/16 |
| Location 6 | 26/28/32 | 22/25/28 | 21/25/28 | 20/25/27 | 19/24/27 | 22/30/36 | 12/23/28 |
| Location 7 | 22/22/22 | 15/16/16 | 15/16/16 | 14/15/15 | 13/14/14 | 21/22/22 | 13/13/13 |

The below tables shows specific turnover time estimations for different operating rooms of a particular redacted facility during actual experimentation.

| | Scheduled Turnover | | Actual Turnover | | Available | Model (2023 |
|---|---|---|---|---|---|---|
| Location 7 | [Second] | | [Second] | | Data | November) |
| name_internal | Mean | Std | Mean | Std | Count | Mean |
| OR01 | 2,630 | 2,583 | 2,754 | 2,352 | 478 | |
| OR02 | 2,829 | 2,837 | 3,129 | 2,728 | 481 | |
| OR05 | 2,808 | 2,627 | 3,823 | 3,032 | 1207 | 3,822 |
| OR06 | 2,709 | 2,332 | 3,651 | 2,736 | 1241 | 3,584 |
| OR07 | 2,491 | 2,283 | 2,700 | 2,131 | 1537 | 2,692 |
| OR08 | 2,651 | 2,489 | 3,096 | 2,626 | 1319 | 3,004 |
| OR09 | 3,722 | 4,974 | 4,431 | 4,631 | 204 | |
| OR10 | 6,012 | 6,792 | 6,677 | 6,672 | 385 | |
| OR11 | 5,361 | 5,548 | 5,844 | 5,516 | 251 | |
| OR12 | 4,178 | 5,146 | 5,146 | 5,279 | 278 | |
| OR14 | 9,399 | 7,467 | 10,290 | 7,554 | 114 | |
| OR15 | 3,002 | 2,757 | 3,250 | 2,670 | 238 | |
| OR17 | 3,818 | 3,614 | 4,583 | 3,600 | 240 | |
| OR18 | 3,473 | 3,354 | 3,967 | 3,104 | 271 | |
| OR19 | 2,713 | 2,519 | 3,495 | 3,149 | 250 | |
| OR20 | 3,117 | 3,289 | 3,448 | 3,457 | 422 | |
| OR21 | 3,051 | 2,462 | 4,038 | 3,332 | 93 | |
| OR22 | 2,282 | 1,752 | 3,042 | 2,405 | 206 | |
| OR24 | 2,711 | 2,021 | 2,899 | 2,416 | 16 | |
| OR25 | 2,706 | 2,476 | 3,586 | 2,239 | 52 | |
| OR26 | 2,996 | 2,700 | 3,805 | 3,082 | 122 | |
| OR27 | 2,214 | 1,672 | 3,011 | 2,188 | 141 | |
| OR28 | 2,370 | 1,827 | 3,216 | 2,489 | 210 | |
| OR29 | 2,196 | 1,648 | 2,586 | 1,874 | 380 | |
| OR30 | 3,387 | 3,994 | 4,315 | 3,985 | 782 | 4,286 |
| OR31 | 3,605 | 2,370 | 3,922 | 2,514 | 11 | |
| OR32 | 5,170 | 5,123 | 5,490 | 5,339 | 274 | |
| OR33 | 4,310 | 4,528 | 4,878 | 4,799 | 276 | |
| OR34 | 6,541 | 6,318 | 6,914 | 6,518 | 62 | |
| OR35 | 10,718 | 9,809 | 11,203 | 8,825 | 64 | |
| OR36 | 11,860 | 10,944 | 11,820 | 10,812 | 62 | |
| OR37 | 3,923 | 4,521 | 4,947 | 4,494 | 122 | |
| OR39 | 2,639 | 2,384 | 3,083 | 2,940 | 232 | |
| OR40 | 2,879 | 1,336 | 3,417 | 1,883 | 190 | |
| OR41 | 2,913 | 3,085 | 3,461 | 2,769 | 195 | |
| OR42 | 3,723 | 4,435 | 4,345 | 4,085 | 138 | |
| OR44 | 2,400 | 1,440 | 2,950 | 1,282 | 6 | |

The scheduling engine can be configured to estimate turnover based on state data, and these can include a first prediction stage (stage 0: before prior wheels_in, stage 1: between prior case wheels_in and wheels_out, and stage 2: after prior case wheels_out).

Gaps between cases can be defined as from T to next case's scheduled_start (T: prior case's observed end or predicted end).

The turnover prediction model implemented follows these processes:
For gaps >1.5 h: Use post case scheduled start as predicted end
For gaps 10 min-1.5 h: Model predicts turnover duration
For gaps <10 min: Default 33-minute turnover time applies

The current model frequently defaults to 33-minute turnovers, especially when rooms fall behind schedule and gaps to the next scheduled cases shrink. In another variation, the approach is configured to explore insights from gaps and other possible features to more accurately define the predicted turnover times.

In a variation, it is proposed that:
For gaps >1.5 h: Use post case scheduled start as predicted end
For gaps <1.5 h: Model predicts turnover duration using gap time and other features

According to the assumption that turnover time mostly relies on prior and post case info, a dataset can be created using each case pair and set observation times for stage 0 (before prior case start), stage 1 (between prior case wheels_in and wheels_out in 15 min intervals), stage 2 (between prior case wheels_out and post case wheels_in in 15 min intervals). 2022 data can be set as a cold start. Using 2023-2024 valid timeline data, the approach included split training and testing data 4:1, with the split date being 2024 Aug. 2. The approach included only samples where the gap time is less than 1.5 hours for modeling.

During experimentation, Applicants constructed potentially relevant features, then eliminated those with low permutation importance and those whose removal would improve MAE, and experimented with 20 features. Each of the 20 features were ranked from a feature importance perspective, and experimentations were conducted with removing different features, and it was found that certain features could be removed: including prior_end_delay, prior_obs_end, prior_pred_start, prior_dur2, . . . , etc., while others could not as their removal would increase MAE.

Ultimately, the model used 14 features in the model, including: Prior case features: prior case scheduled start, prior case start, prior case start delay (negative if start early), prior case scheduled end, prior case end, prior case scheduled duration, prior case duration (predicted duration/actual duration); Post case features, post case scheduled start, post case scheduled end, post case scheduled duration; Duration features: turnover scheduled duration, gap; Elapsed time feature: turnover elapsed, Surgeon feature: is prior case main surgeon same as post case main surgeon.

From SHAP values, Applicants observed several patterns: Longer values in the following features contribute to longer predicted durations (scheduled turnover duration, post case scheduled duration, prior case scheduled duration, turnover elapsed time, gap); Surgeon transitions impact turnover time (same surgeon leads to shorter turnovers) and Earlier prior case end times correlate with longer predicted durations.

Despite apparent redundancy among some features (e.g., scheduled duration versus scheduled start/end), removing any of these 14 features increased cross validation MAE, so the model was configured ultimately to retain them all.

The following table shows the MAE increases for removing various features:

| Feature Name | MAE Increase(s) |
|---|---|
| turnover_elapsed | 52.0 |
| is_same_surgeon | 31.4 |
| post_scheduled_duration | 13.1 |
| turnover_scheduled_duration | 12.5 |
| post_scheduled_start | 5.8 |
| prior_start_delay | 5.0 |
| post_scheduled_end | 4.2 |
| gap | 3.4 |
| prior_scheduled_start | 2.4 |
| prior_scheduled_duration | 2.3 |
| prior_end | 2.0 |
| prior_acutal_duration | 1.5 |
| prior_start | 1.4 |
| prior_scheduled_end | 0.3 |

Applicants compared both linear and tree-based models using the current feature set with non-null values. Tree models outperformed linear models. Among tree-based models, LightGBM demonstrated the best performance.

In experimentation, the new model performs better than the 33-minute baseline and an older model at Location 1, but shows lower performance at Locations 2 and 3.

Analysis of Location 3 cases shows actual durations are close to 33 minutes, but the LightGBM model predicts longer times due to its reliance on scheduled turnover duration. While this creates some discrepancies, scheduled turnover duration remains an important feature for overall model performance, and these cases represent a minor subset.

After fitting the model, it was used to build new timelines and evaluate overall performance. The LightGBM model demonstrates improved performance across all facilities.

As described herein in some embodiments, the system is also configured to automatically determine one or more future room state configurations to be proposed as candidate options for selection by the administrator, who can implement the room state configurations for future scheduling through selecting from a series of options being presented. In another embodiment, the system can automatically operate and re-arrange and dynamically schedule operations for the future based on the existing room state and/or determinations of future room state.

In some embodiments, room state modifications for future estimations are conducted using a machine learning model that takes into account previous room state determinations associated with a particular room or surgical team, adding time modifications (e.g., expected delays). For example, a particular surgeon may be very experienced, and typically ends surgeries early. This can be taken into account during dynamic rescheduling. The simulations can be used for dynamic rescheduling, providing estimated durations that can then be utilized for schedule optimization/modification.

The scheduling optimization can be conducted, for example, using the CP-SAT Solver, a tool that can be practically used for schedule optimization tasks. A number of different assumptions and constraints can be generated for a particular facility, such as set up and cleanup times, rest periods, specialty room assignment robotic case assignments, among others.

The CP-SAT Solver can be given a specific objective function to optimize for, based on a weighted combination of factors such as idle time, overtime, unused room time, number of cases modified (e.g., number of cases moved from one room to another against an original schedule). The CP-SAT Solver output can be run using the room state predictions as an input, as well as an original schedule and other information obtained from the various EMRs, as noted below.

The CP-SAT Solver output can be used to re-generate periodically an optimized schedule, or in some embodiments, the CP-SAT Solver can be triggered whenever a major perturbation or scheduling event has occurred (e.g., a threshold number of unexpected cases have arisen, a case state is being tracked far in excess or below the predicted time). Having a case being tracked to finish far earlier than a predicted time provides the CP-SAT Solver an opportunity to opportunistically improve a future schedule, which may be useful, for example, to find a solution that has a large impact on overtime reduction or improving utilization, for example, freeing up resources for other patients and improving the overall quality of care.

The CP-SAT Solver predicted outputs can be provided in the form of a data structure having assigned timeslots for various procedures, such as a series of individual ICS files generated for each procedure that are then distributed to control the calendaring of one or more individuals, such as surgeons, hospital staff, and room/machine bookings. In some embodiments, the CP-SAT Solver is configured to “lock in” and no longer modify bookings within a period of time to avoid requesting or rescheduling difficult to move resources. In some embodiments, the ICS files are only sent once the “lock in” duration is reached to avoid assigning and reassigning resources with every dynamic rescheduling.

In dynamic rescheduling, the system can be provided with an objective function of minimizing overtime and idle time, and optimizations are run in real-time that result in an expected decrease in idle time and/or overtime. The mechanism to allow this to happen is that since the system already has insight into the expected overtime and idle time of each operating room, the system can identify for example the room at most risk for overtime, and the room with the most expected idle time.

In hospital setting, each operation room has their scheduled block time to schedule cases. For example, 7 AM to 5 PM. If any case scheduled within the window but operated outside the window, this is defined as overtime. If cases are operated outside the window, the cost are higher than during regular times. Therefore, if a set of methods can help hospital to 1) build a accurate schedule, 2) have a reminder if the chance of overtime increased, the hospital can actively manage the schedule to reduce the cost.

An overtime probability prediction engine using a trained model is proposed.

For estimating overtime, the approach can include using the model to generate an active prediction, and during experimentation, Applicants choose these 8 features for predicting active probability: First segment predicted or observed start, Last case predicted end, Difference between last case predicted end and target (Overtime), Clipped overtime divided by remaining operation time, Clipped overtime divided by remaining case time, Remaining operation time, Remaining case time, Remaining N cases. All times were normalized by seconds in a day.

Applicants tested a number of different models, and it was found that The GLM with a Bernoulli family and Log log link function overall performed the best, with the lowest log loss. The results demonstrates that the model is effectively capturing the relationship between the predicted end time and the active probability at the target time.

Experimental results are provided below:

| | | ROC | PR | | | |
|---|---|---|---|---|---|---|
| | Models | AUC | AUC | log loss | brier | Accuracy |
| Binary | Logistic Regression | 0.9346 | 0.9243 | 0.3954 | 0.1272 | 0.8524 |
| Classification | GLM Logit link + | 0.9347 | 0.9245 | 0.3947 | 0.1270 | 0.8523 |
| | bernoulli | | | | | |
| | GLM Probit link + | 0.9347 | 0.9244 | 0.3989 | 0.1273 | 0.8521 |
| | bernoulli | | | | | |
| | GLM Cauchy link + | 0.9347 | 0.9244 | 0.4034 | 0.1285 | 0.8533 |
| | bernoulli | | | | | |
| | GLM Loglog link + | 0.9347 | 0.9244 | 0.3894 | 0.1266 | 0.8537 |
| | bernoulli | | | | | |
| Regression | GLM identity + | 0.9344 | 0.9241 | 0.4033 | 0.1287 | 0.8526 |
| (compute | gaussian | | | | | |
| P(y > 0)) | GLM identity + | 0.9344 | 0.9241 | 0.4033 | 0.1287 | 0.8526 |
| | skewed norm | | | | | |
| | GLM identity + t | 0.9344 | 0.9241 | 0.3972 | 0.1277 | 0.8535 |

Further testing yielded the following results: They have similar performance, Logistic regression overall performed the best, with the lowest log loss and highest accuracy.

| Models | ROC AUC | PR AUC | log loss | brier | Accuracy |
|---|---|---|---|---|---|
| Logistic | 0.935 | 0.930 | 0.324 | 0.101 | 0.859 |
| Regression | | | | | |
| GLM Logit | 0.935 | 0.930 | 0.324 | 0.101 | 0.858 |
| link + | | | | | |
| bernoulli | | | | | |
| GLM | 0.935 | 0.930 | 0.326 | 0.101 | 0.858 |
| Probit link + | | | | | |
| bernoulli | | | | | |
| GLM | 0.935 | 0.929 | 0.334 | 0.103 | 0.859 |
| Cauchy | | | | | |
| link + | | | | | |
| bernoulli | | | | | |
| GLM | 0.935 | 0.929 | 0.324 | 0.101 | 0.858 |
| Loglog | | | | | |
| link + | | | | | |
| bernoulli | | | | | |

The system can then generate recommendations (or control commands) moving a case from the overtime room to the idle time, thus lowering both expected overtime and idle time. There are additional constraints surrounding staff, resource, and potential geographic constraints within the hospital that need to be taken into consideration during re-scheduling or optimization, and these are factors that can be incorporated into the re-scheduling engine.

Similar to the Turnover Modeling approach, the elapsed time of a case also offers valuable insights for predicting model duration. This concept bears resemblance to a Bayesian approach, wherein the duration of a case is conditioned on the time already elapsed. In a proposed implementation of machine learning feature engineering, the system can introduce an additional feature that signifies the ratio of elapsed time to the scheduled time. The elapsed time of a case can further be based on an internal procedure map, as well as additional information from an EMR or a schedule, such as whether a procedure was robot assisted, what type of assistance robot was used, among others, which provides another useful feature for increased elapsed time determination.

Below, a non-limiting example showcasing cases at different timestamps is provided.

| | Scheduled | Actual | |
|---|---|---|---|
| | Time | Duration | Elapsed Ratio |
| Case ID | [minute] | [minute] | [equal to time at] |
| A | 60 | 90 | 0.0 [0 minute] |
| A | 60 | 90 | 0.1 [6 minute] |
| A | 60 | 90 | 0.5 [30 minute] |
| A | 60 | 90 | 1.0 [60 minute] |
| A | 60 | 90 | 1.25 [75 minute] |

In addition to implementing this adjustment, one must also revise the target encoding criteria to accommodate the conditional approach. Specifically, the approach needs to exclude cases that ended prior to a certain percentage of the scheduled time and calculate the average duration of the remaining cases accordingly. Additionally, a method for grouping cases with significantly different scheduled times separately during the encoding process is required. For instance, it is necessary to distinguish between cases scheduled for 1 hour and those scheduled for 10 hours. This adjustment applies to both facility ID and surgeon ID encoding.

To ensure the model can accurately predict all different procedures without being adversely affected by relatively low data availability, a mask is proposed for masking all features related to procedure-level information with unknown values. This includes features such as procedure level average duration, among others. Once the BBP procedure map becomes available, the approach can then leverage the procedure table to populate these missing values.

It was found that the model consistently exceeds the scheduled time across all facilities. A significant enhancement in model performance is observed at approximately the 90% threshold across all facilities. However, for two locations, the model performs below the scheduled time after incorporating these conditional features when the elapsed time is less than 100%. This discrepancy may be attributed to the highly variable actual durations, which diminishes the utility of these features in these specific scenarios and consequently undermines the model's performance for these facilities.

In some embodiments, the decision support interface can also be used to generate notifications. Currently, when there are scheduling changes or delays in the operating room schedule throughout the day, this information either is not communicated, or it is communicated to the staff by an operating nurse via a phone call. This feature attempts to automate the need for staff to send out these notifications, thus increasing the risk for human error in getting this information to relevant parties. For example, if a case gets delayed by 1 hr, an event trigger is configured that sends a notification to all participants in that case that the case is delayed.

Once the system has produced a timeline and associated distributions for each predicted state, this data object is stored in a REDIS™ cache. Since this simulation is expensive to run, it may not necessarily be run every time there is new information (which is every 60 seconds in the example above). This would require a level of computation power beyond what allows for seamless scalability. Therefore, the system can be configured to only re-run the simulation if a new state is observed.

Another mechanism for which the system can be configured for changing a timeline is if a room is still in the same state as the previous observation, but this state is expected to finish within 5 minutes. If this is the case, the system can shift the entire timeline to later. This prevents a situation where the predicted timeline is out of sync with the actual state.

In order to preserve model performance and prevent model drift, ongoing retraining can occur, where the system is configured to retrain the deep learning and smoothing models in an ongoing and automated way. Each month, for example, human analysts can annotate data. At the end of the month, a retraining computational job is automatically triggered. Of all the annotated videos, the job determines the set of videos for which the model performed the worst. It sets these aside as the validation set. The job then retrains the model on the other videos to produce a new model. Finally, the job evaluates the performance of the new model against a fixed test set that remains constant month to month. If the performance of the new model is superior, the model is automatically inserted into the workflow.

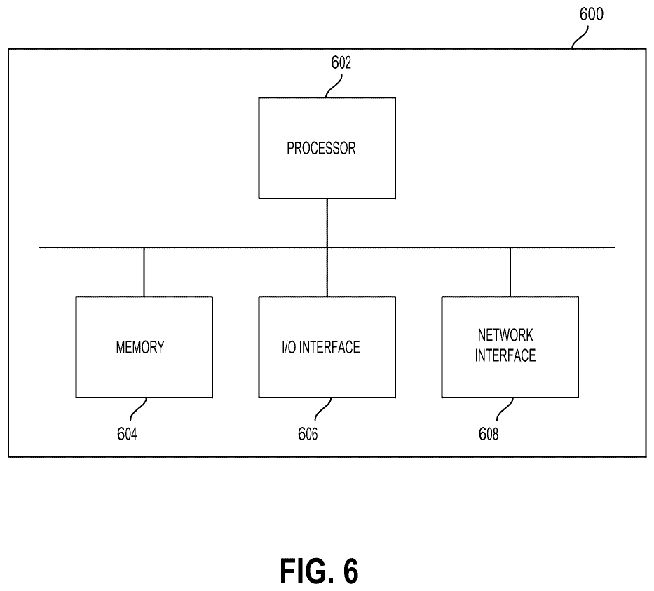

FIG. 6 is an example computing system diagram 600 showing an example practical computing system for implementing the system for room state determination using machine vision, according to some embodiments.

The computing device 600 can be a computer server or other physical computing hardware device, and may reside, for example, in a data center. The computing device 600 includes one or more computer processors (e.g., microprocessors) 602 which are adapted to execute machine interpretable instructions, and interoperates with computer memory 604 (e.g., read only memory, random access memory, integrated memory). An input/output interface 604 can be provided that receives data sets representing inputs from devices such as computer mice, keyboards, touch screens, among others, and provides outputs in the form of interface element control for rendering on computer displays, such as monitors. A network interface 606 is provided that is adapted for electronic communications with other computing devices, such as downstream computing systems, data storage elements, data backup servers, among others. The network interface 606 can include various types of interfaces, including wireless connection interfaces, wired connection interfaces, connections with messaging buses, among others.

Applicant notes that the described embodiments and examples are illustrative and non-limiting. Practical implementation of the features may incorporate a combination of some or all of the aspects, and features described herein should not be taken as indications of future or existing product plans.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present application is not intended to be limited to the particular embodiments of the process, machine, manufacture, composition of matter, means, methods and steps described in the specification.

As one will readily appreciate from the disclosure, processes, machines, manufacture, compositions of matter, means, methods, or steps, presently existing or later to be developed, that perform substantially the same function or achieve substantially the same as the corresponding embodiments described herein may be utilized. Accordingly, the appended embodiments are intended to include within their scope such processes, machines, manufacture, compositions of matter, means, methods, or steps.

As can be understood, the examples described above and illustrated are intended to be exemplary only.

What is claimed is:

1. A system for room state determination using machine vision, the system comprising:
a computer processor operating in conjunction with computer memory and non-transitory computer readable storage media, the computer processor configured to:
receive, from one or more video recording devices capturing video streams including a plurality of time-stamped video frames relating to a corresponding room;
pre-process the one or more video streams using a first inference model to conduct deep learning inference to process the time-stamped video frames to predict a set of first room state predictions, each for a particular time duration or point in time based on the frames at the time duration or the particular point in time;
process the intermediate room state using a second smoothing model to generate a second set of room state predictions based on the first set of room state predictions;
process the second set of intermediate room state predictions using a third flicker reduction model to generate a third set of room state predictions; and
provide the third set of room state predictions to a downstream computing interface or system for user interface generation or for input into a scheduling computer process.

2. The system of claim 1, wherein the first inference model is a shufflenet convolutional neural network (CNN) that is configured to utilize transfer learning.

3. The system of claim 2, wherein the second smoothing model is based at least on XGBoost, and wherein a feature being configured on XGBoost include at least a number of past frames being used for inference, a past time duration, or a time of day.

4. The system of claim 3, wherein the second smoothing model is further configured to develop a deep learning output based on separate deep learning outputs corresponding to each camera of a plurality of cameras, and to develop the second set of room state predictions based on a combination of each of the separate deep learning outputs corresponding to each camera of the plurality of cameras.

5. The system of claim 1, wherein the third flicker reduction model is configured to limit a change in predicted state to only scenarios where the first, second, or third models have a confidence score greater than a threshold for a prediction, or where a prediction state change, despite having the confidence score lower than the threshold for a prediction, has persisted for a pre-defined number of previous frames.

6. The system of claim 1, wherein the third set of room state predictions are utilized to generate an initial snapshot of current predicted room states at a particular point in time.

7. The system of claim 6, wherein the third set of room state predictions are utilized to extrapolate for future predicted room states based on identified scheduling dependencies based on a simulation of an existing schedule.

8. The system of claim 6, wherein the third set of room state predictions are utilized to extrapolate for future predicted room states based on identified scheduling dependencies based on a plurality of simulations based on different future perturbations of an existing schedule.

9. The system of claim 8, wherein for each of the plurality of simulations, an operational parameter is determined, and a target simulation and scheduling configuration is determined as an optimal configuration, and wherein a scheduling process automatically conducts rescheduling to match the optimal configuration.

10. The system of claim 1, wherein the system for room state determination using machine vision is implemented on cloud computing resources on distributed computing resources.

11. A method for room state determination using machine vision, the method comprising:
receiving, from one or more video recording devices capturing video streams including a plurality of time-stamped video frames relating to a corresponding room;
pre-processing the one or more video streams using a first inference model to conduct deep learning inference to process the time-stamped video frames to predict a set of first room state predictions, each for a particular time duration or point in time based on the frames at the time duration or the particular point in time;
processing the intermediate room state using a second smoothing model to generate a second set of room state predictions based on the first set of room state predictions;
processing the second set of intermediate room state predictions using a third flicker reduction model to generate a third set of room state predictions; and
providing the third set of room state predictions to a downstream computing interface or system for user interface generation or for input into a scheduling computer process.

12. The method of claim 11, wherein the first inference model is a shufflenet convolutional neural network (CNN) that is configured to utilize transfer learning.

13. The method of claim 12, wherein the second smoothing model is based at least on XGBoost, and wherein a feature being configured on XGBoost include at least a number of past frames being used for inference, a past time duration, or a time of day.

14. The method of claim 13, wherein the second smoothing model is further configured to develop a deep learning output based on separate deep learning outputs corresponding to each camera of a plurality of cameras, and to develop the second set of room state predictions based on a combination of each of the separate deep learning outputs corresponding to each camera of the plurality of cameras.

15. The method of claim 11, wherein the third flicker reduction model is configured to limit a change in predicted state to only scenarios where the first, second, or third models have a confidence score greater than a threshold for a prediction, or where a prediction state change, despite having the confidence score lower than the threshold for a prediction, has persisted for a pre-defined number of previous frames.

16. The method of claim 11, wherein the third set of room state predictions are utilized to generate an initial snapshot of current predicted room states at a particular point in time.

17. The method of claim 16, wherein the third set of room state predictions are utilized to extrapolate for future predicted room states based on identified scheduling dependencies based on a simulation of an existing schedule.

18. The method of claim 16, wherein the third set of room state predictions are utilized to extrapolate for future predicted room states based on identified scheduling dependencies based on a plurality of simulations based on different future perturbations of an existing schedule.

19. The method of claim 18, wherein for each of the plurality of simulations, an operational parameter is determined, and a target simulation and scheduling configuration is determined as an optimal configuration, and wherein a scheduling process automatically conducts rescheduling to match the optimal configuration.

20. A non-transitory computer readable medium, which when executed by a processor, cause the processor to perform steps of a method for room state determination using machine vision, the method comprising:
receiving, from one or more video recording devices capturing video streams including a plurality of time-stamped video frames relating to a corresponding room;
pre-processing the one or more video streams using a first inference model to conduct deep learning inference to process the time-stamped video frames to predict a set of first room state predictions, each for a particular time duration or point in time based on the frames at the time duration or the particular point in time;
processing the intermediate room state using a second smoothing model to generate a second set of room state predictions based on the first set of room state predictions;
processing the second set of intermediate room state predictions using a third flicker reduction model to generate a third set of room state predictions; and
providing the third set of room state predictions to a downstream computing interface or system for user interface generation or for input into a scheduling computer process.
